(19) United States
(12) Patent Application Publication
(10) Pub. No.: US 2011/0118706 A1
(43) Pub. Date: May 19, 2011

(54) Device Suitable for Use During Deployment of a Medical Device

(75) Inventors: Peter Gingras, Galway (IE); Dean King, Galway (IE)

(73) Assignee: PROXY BIOMEDICAL LIMITED, Galway (IE)

(21) Appl. No.: 12/675,943

(22) PCT Filed: Sep. 1, 2008
(86) PCT No.: PCT/EP08/61515

§ 371 Date: Jan. 21, 2011

(30) Foreign Application Priority Data
Aug. 30, 2007 (IE) 2007/0623

(51) Int. Cl. A61B 17/00

(52) U.S. Cl. 606/1

(57) ABSTRACT

A medical assembly comprises a soft tissue implant (16) for treating a first portion of body tissue during hernia repair, and a device (20). The device (20) comprises a support element (31) to support the soft tissue implant (16) during deployment. The device (20) comprises an elongate drawstring to releasably couple the soft tissue implant (16) to the support element (31). The support element (31) and the soft tissue implant (16) are movable between a collapsed delivery configuration, and an expanded deployment configuration. During deployment a second end (33) of the support element (31) engages with a second portion of body tissue to maintain the second portion of body tissue spaced-apart from the soft tissue implant (16). The support element (31) comprises an access opening (34) through which one or more instruments may be extended to access the soft tissue implant (16). The soft tissue implant (16) is attached to the first portion of body tissue using a suture. After deployment the support element (31) may be released from the soft tissue implant (16) and removed.

### INTRODUCTION

This invention relates to a device suitable for use during deployment of a medical device at a desired treatment location.

It is known to use soft tissue implants to reinforce or replace areas of the human body that have acquired defects. These implants may require invasive means of delivery, which may result in complications and longer recovery periods for the patient.

### STATEMENTS OF INVENTION

According to the invention there is provided a device suitable for use during deployment of a medical device at a desired treatment location, the device comprising a support element to support the medical device during deployment.

In one embodiment of the invention the support element comprises a first end mounted to a medical device. Preferably the support element comprises a second end longitudinally spaced-apart from the first end. Ideally the second end of the support element is engagable with a body tissue to maintain the body tissue spaced-apart from a medical device. By maintaining the body tissue spaced-apart from the medical device, this provides the clinician with sufficient working space to attach the medical device at the desired treatment location. Most preferably the support element is configured for location fully within an internal body cavity. The second end of the support element may be insertable into an internal body cavity. Preferably the support element tapers inwardly from the first end towards the second end. Ideally the support element is curved in longitudinal cross-section. Most preferably the concave portion of the curve faces radially outwardly. The support element may be substantially dome-shaped.

In another embodiment the support element comprises an access opening extending at least partially therethrough through which one or more parts may be extended to access a medical device. The part extended through the access opening may be an instrument or a hand/arm of a clinician. Preferably the access opening is located substantially at the second end of the support element. Ideally the access opening is located substantially at the radial centre of the support element.

In one case the device comprises a coupling element to couple a medical device to the support element. Preferably the coupling element is configured to releasably couple a medical device to the support element. By releasing the support element from the medical device, this enables the support element to be removed after the medical device has been attached at the desired treatment location. Ideally the coupling element is extendable through an opening in the support element. Most preferably the coupling element is extendable through an opening in a medical device. The coupling element may be substantially elongate. Preferably the coupling element comprises a drawstring. Ideally the coupling element comprises a low-friction material.

In another case the support element is movable between a delivery configuration for delivery of a medical device to a desired treatment location, and a deployment configuration for deployment of the medical device at the desired treatment location. Preferably the support element is collapsed in the delivery configuration. This low profile provides for ease of delivery. Ideally the support element is expanded in the deployment configuration. Most preferably the support element is biased towards the deployment configuration. The material of the support element may provide the required outward force to deploy the support element and the medical device. The support element may comprise a resilient material. Preferably the support element comprises a shape-memory material. Ideally the support element comprises a thermoplastic material. The support element may comprise a bioabsorbable material.

The invention also provides in another aspect an assembly comprising:

a medical device; and
a device of the invention for use during deployment of the medical device at a desired treatment location.

In one embodiment of the invention the medical device comprises an implant. Preferably the medical device comprises a soft tissue implant.

In another embodiment the medical device is configured to be attached to a body tissue at a desired treatment location. Preferably the medical device comprises one or more openings through which an attachment element may be extended. Ideally the medical device comprises one or more openings through which the coupling element is extendable.

In a further aspect of the invention there is provided a method of deploying a medical device at a desired treatment location, the method comprising the steps of:

using a support element to support the medical device during deployment at the desired treatment location,
attaching the medical device to a first body tissue at the desired treatment location, and
removing the support element from the desired treatment location.

The support element may be removed by withdrawing the support element from the desired treatment location. Alternatively the support element may be provided in the form of a bioabsorbable element, and the support element may be removed by allowing the support element to bioabsorb.

In one embodiment of the invention during deployment of the medical device at the desired treatment location, the support element engages with a second body tissue to maintain the second body tissue spaced-apart from the medical device. By maintaining the second body tissue spaced-apart from the medical device, this provides the clinician with sufficient working space to attach the medical device at the desired treatment location.

In another embodiment one or more parts are used to attach the medical device to the first body tissue. The part used to attach the medical device to the first body tissue may be an instrument or a hand/arm of a clinician. Preferably the method comprises the step of extending the part at least partially through the support element to access the medical device.

In one case the method comprises the step of coupling the medical device to the support element before delivery to the desired treatment location. Preferably the method comprises the step of de-coupling the medical device from the support element after attaching the medical device to the first body tissue. By releasing the support element from the medical device, this enables the support element to be removed after the medical device has been attached at the desired treatment location.

In another case the method comprises the step of collapsing the medical device before delivery to the desired treatment location. This low profile provides for ease of delivery. Preferably the method comprises the step of expanding the medical device before attaching to the first body tissue.

The invention also provides in another aspect a soft tissue implant for repairing a bodily defect comprising:

a first shape memory delivery system component sized and shaped to extend beyond the bodily defect in a patient, the delivery system having a domed configuration and having a selected elasticity,
a biocompatible soft tissue implant; and
an attachment means to the soft tissue implant, the attachment means being reversibly attached to the soft tissue implant.

In one case the invention provides a minimally invasive delivery system for soft tissue implants.

In another case the invention provides a soft tissue implant and method for making the same.

In one case the invention provides an improved delivery system for deploying soft tissue implants for treating bodily defects.

In another case the invention provides improved soft tissue implants and methods of soft tissue implant delivery, specifically implants that treat bodily defects in a minimally invasive fashion.

The invention has a number of advantages over known approaches.

The invention provides an implant and delivery means that results in a procedure with a smaller surgical site incision, with a decrease in pain and shorter recovery periods for the patient.

The invention provides a reduction in postoperative wound healing complications, such as infections and seroma formation, which is correlated with smaller incisions, less trauma and dead space between the prosthesis and the host tissues.

The invention provides an implant and delivery means that results in a procedure that allows for predictable expansion and securement of the implant which may result in decreased recurrence rates and patient discomfort.

The invention provides an implant and delivery means that results in a procedure that allows for shorter procedure times and does not require general anaesthesia which decreases the risk of procedural complications.

The invention provides an implant and delivery means that results in decreased material content, and that results in a flexible implant with a reduced inflammatory response, and physical properties of the implant that simulate the physical properties of the tissue structures being replaced.

The invention provides an implant and delivery means that permits adequate visualisation of the implant and creates a working space for fixation of the implant during the procedure.

The invention provides a soft tissue implant and delivery means, which has an outstanding combination of ease of delivery and low residual material content.

The invention provides a soft tissue implant and delivery means that provides enhanced placement and biocompatibility in a low profile configuration while maintaining the requisite strength to repair tissue.

In one case the invention provides a minimally invasive delivery system for soft tissue implants for treating bodily defects comprising an elastic and detachable delivery system attached to a soft tissue implant.

In another case, the invention provides a delivery system and soft tissue implant in a biocompatible form. The implant may have a structure characterised by a dome reversibly attached to a soft tissue implant using a drawstring means. The delivery system may be designed to optimise expansion and working space in the subcutaneous tissue space to facilitate soft tissue implant attachment. The implant material may have high flexibility, high strength, high porosity, and a low surface area. The soft tissue implant and delivery means may provide an outstanding combination of ease of delivery and low residual material content.

### BRIEF DESCRIPTION OF THE DRAWINGS

The invention will be more clearly understood from the following description of some embodiments thereof, given by way of example only, with reference to the accompanying drawings, in which:

FIG. 1 is a micrograph of a known polypropylene mesh.

FIG. 2 is a perspective view of a film in accordance with the invention.

FIG. 3A is a plan view of a sinusoidal like cell pattern machined in the film with major and minor struts in accordance with the invention.

FIG. 3B is a plan view of a sinusoidal like cell pattern machined in the film with delivery system attachment points in accordance with the invention.

FIG. 4A is a perspective view of a minimally invasive delivery system in accordance with the invention.

FIG. 4B is a top plan view of a minimally invasive delivery system in accordance with the invention.

FIG. 4C is a bottom plan view of a minimally invasive delivery system in accordance with the invention.

FIG. 4D is a side view of a minimally invasive delivery system in accordance with the invention.

FIG. 5A is a perspective view of a minimally invasive delivery system and a soft tissue implant in accordance with the invention.

FIG. 5B is a perspective view of a minimally invasive delivery system attached to a soft tissue implant with a drawstring in accordance with the invention.

FIG. 6A is a perspective view of a minimally invasive delivery system in accordance with the invention.

FIG. 6B is a top plan view of a minimally invasive delivery system in accordance with the invention.

FIG. 6C is a bottom plan view of a minimally invasive delivery system in accordance with the invention.

FIG. 6D is a side view of a minimally invasive delivery system in accordance with the invention.

FIG. 6E is a plan view of an inguinal soft tissue implant in accordance with the invention with a spermatic cord flap.

FIG. 6F is a plan view of an inguinal soft tissue implant in accordance with the invention with a spermatic cord flap positioned over the main body of the implant.

FIG. 7A is a side view of a minimally invasive delivery system in accordance with the invention.

FIG. 7B is a side view of a minimally invasive delivery system in accordance with the invention.

FIG. 7C is a bottom plan view of an inguinal soft tissue implant in accordance with the invention.

FIG. 7D is a plan view of an inguinal soft tissue implant in accordance with the invention.

FIG. 8A is a side view of a minimally invasive delivery system in accordance with the invention.

FIG. 8B is a side view of a minimally invasive delivery system in accordance with the invention.

FIG. 8C is a bottom plan view of an inguinal soft tissue implant in accordance with the invention.

FIG. 9A is a photograph of a patient trainer used for surgical training with a 4 cm incision line.

FIG. 9B is a photograph of a patient trainer used for surgical training with a scalpel making a 4 cm incision.

FIG. 9C is a photograph of a patient trainer used for surgical training with a simulated hernia sac dissection taking place.

FIG. 9D is a photograph of a patient trainer used for surgical training with a minimally invasive delivery system in accordance with the invention.

FIG. 9E is a photograph of a patient trainer used for surgical training with a minimally invasive delivery system in accordance with the invention being placed over the hernia defect.

FIG. 9F is a photograph of a patient trainer used for surgical training with a minimally invasive delivery system in accordance with the invention being expanded to create working space over the hernia defect.

FIG. 9G is a photograph of a patient trainer used for surgical training with a minimally invasive delivery system in accordance with the invention. The surgical mesh attached to the minimally invasive delivery system is being stapled to the tissue using a disposable stapler.

FIG. 9H is a photograph of a patient trainer used for surgical training with the drawstring being cut on the minimally invasive delivery system in accordance with the invention.

FIG. 9I is a photograph of a patient trainer used for surgical training with the drawstring being removed from the minimally invasive delivery system in accordance with the invention.

FIG. 9J is a photograph of a patient trainer used for surgical training with the minimally invasive delivery system being removed from the patient in accordance with the invention.

FIG. 9K is a photograph of a patient trainer used for surgical training with the minimally invasive delivery system removed from the patient in accordance with the invention.

FIG. 9L is a photograph of a patient trainer used for surgical training with the stapled mesh attached to the patient's tissue.

FIG. 10 is a diagram showing the manufacturing steps.

Some reference numerals used in the drawings are as follows:
10 known mesh
12 bio compatible film
14 machined film with sinusoidal cell pattern
16 machined film with sinusoidal cell pattern and attachment points
18 attachment points
20 minimally invasive delivery system dome
22 drawstring attachment means
24 minimally invasive delivery system inguinal dome
26 inguinal soft tissue implant
28 spermatic cord flap
30 fixation attachment point
31 support element
32 support element first end
33 support element second end
34 access opening
35 support element openings

### DETAILED DESCRIPTION

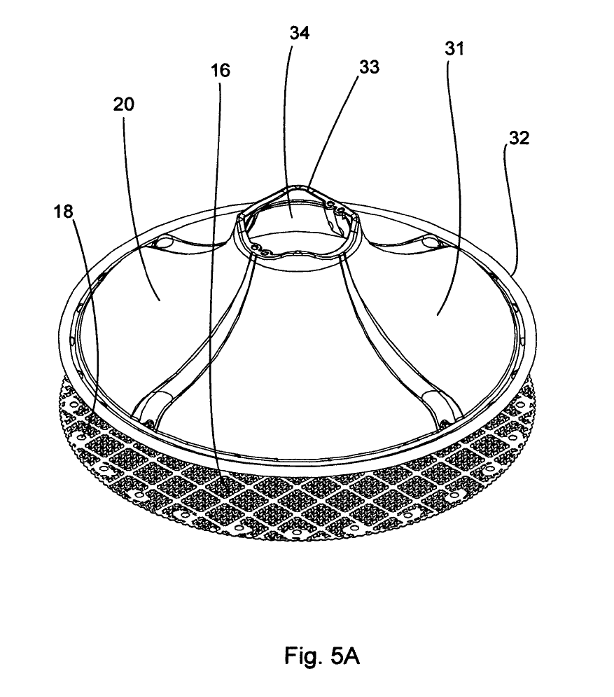

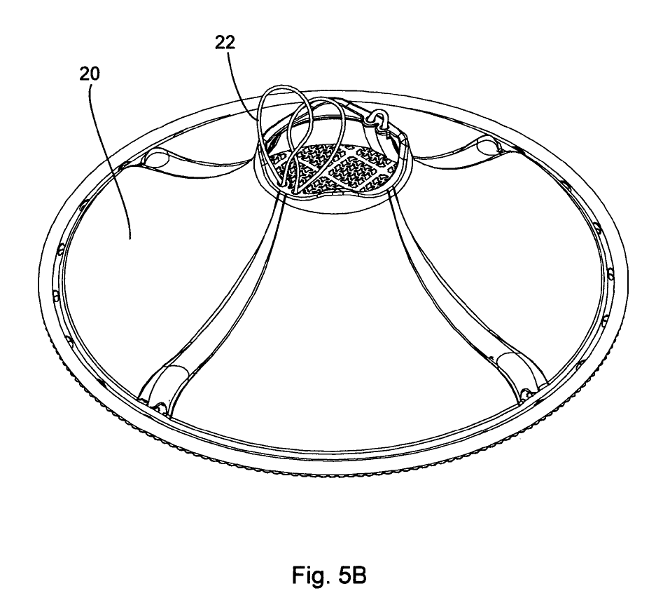

Referring to the drawings, and initially to FIGS. 5A and 5B thereof, there is illustrated a medical assembly according to the invention. The assembly comprises a medical device 16 and a device 20 suitable for use during deployment of the medical device 16 at a desired treatment location.

In this case the medical device 16 comprises a soft tissue implant. The soft tissue implant 16 may be employed to treat a first portion of body tissue during hernia repair. The soft tissue implant 16 may be attached to the first portion of body tissue at the desired treatment location using an attachment element, such as a suture. The soft tissue implant 16 comprises a plurality of openings 18 through which the attachment element may be extended to attach the soft tissue implant 16 to the first portion of body tissue.

The device 20 comprises a support element 31 to support the soft tissue implant 16 during deployment.

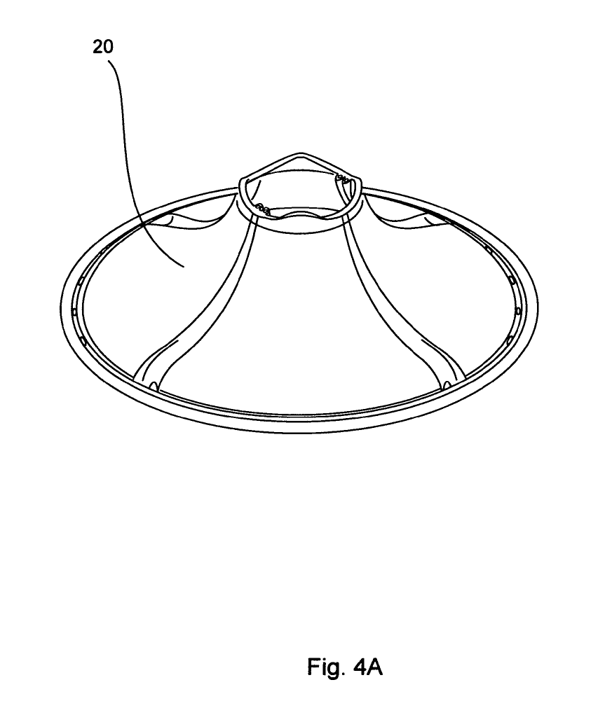

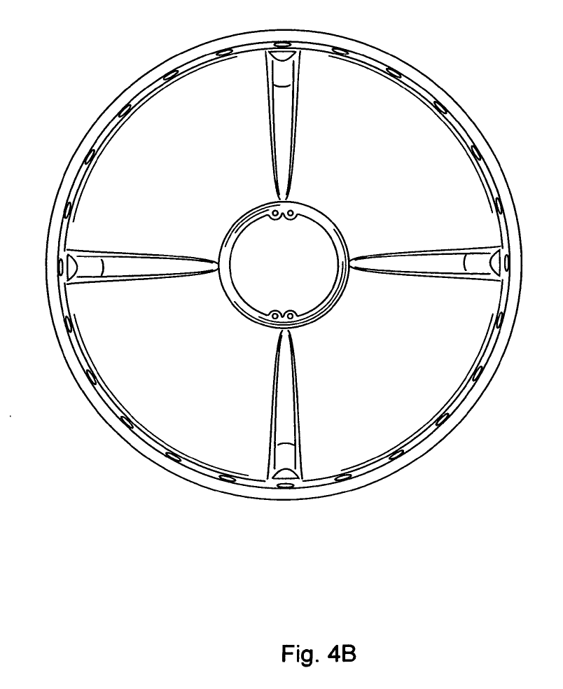

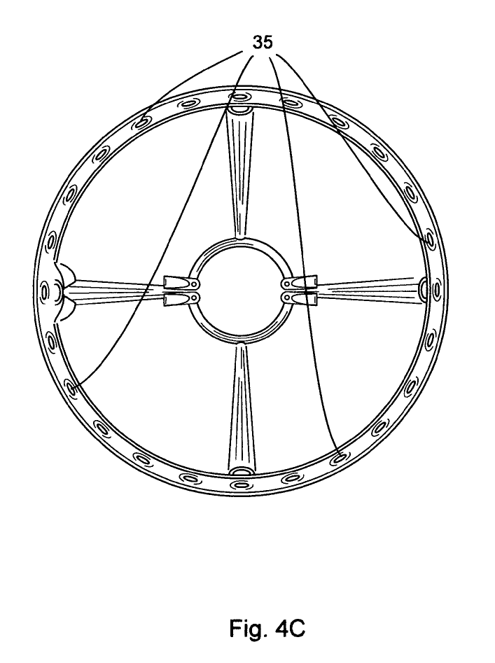

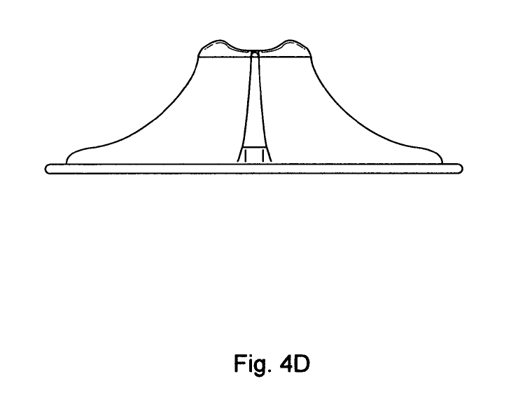

The support element 31 comprises a first end 32 for mounting to the soft tissue implant 16, and a second end 33 longitudinally spaced-apart from the first end 32. The support element 31 tapers inwardly from the first end 32 towards the second end 33. As illustrated in FIG. 4D, the support element 31 is curved in longitudinal cross-section with the concave portion of the curve facing radially outwardly. The support element 31 is substantially dome-shaped (FIG. 4D).

The device 20 comprises a coupling element 22 to releasably couple the soft tissue implant 16 to the support element 31. The coupling element 22 is extendable through a plurality of openings 35 in the support element 31 (FIG. 4C). The soft tissue implant 16 comprises a plurality of openings through which the coupling element 22 is extendable to couple the soft tissue implant 16 to the support element 31.

In this case the coupling element 22 is provided in the form of an elongate drawstring of a low-friction material.

The support element 31 and the soft tissue implant 16 are movable between a collapsed delivery configuration for delivery of the soft tissue implant 16 to the desired treatment location, and an expanded deployment configuration for deployment of the soft tissue implant 16 at the desired treatment location.

The support element 31 is biased towards the deployment configuration. In this manner the support element 31 causes expansion of the soft tissue implant 16 at the desired treatment location. The support element 31 may be provided in the form of a resilient material, and/or a shape-memory material, and/or a thermoplastic material. The support element 31 may comprise a bioabsorbable material.

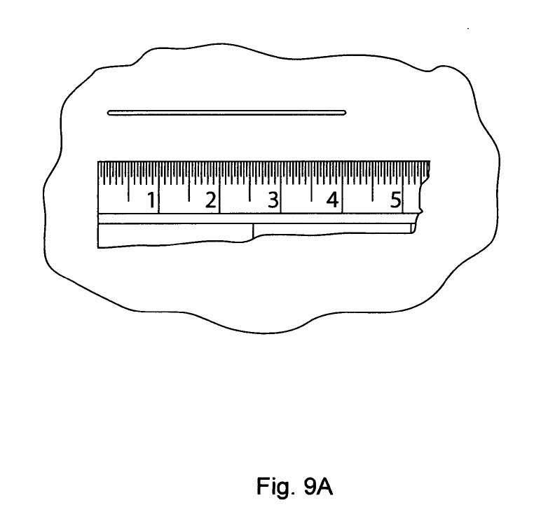

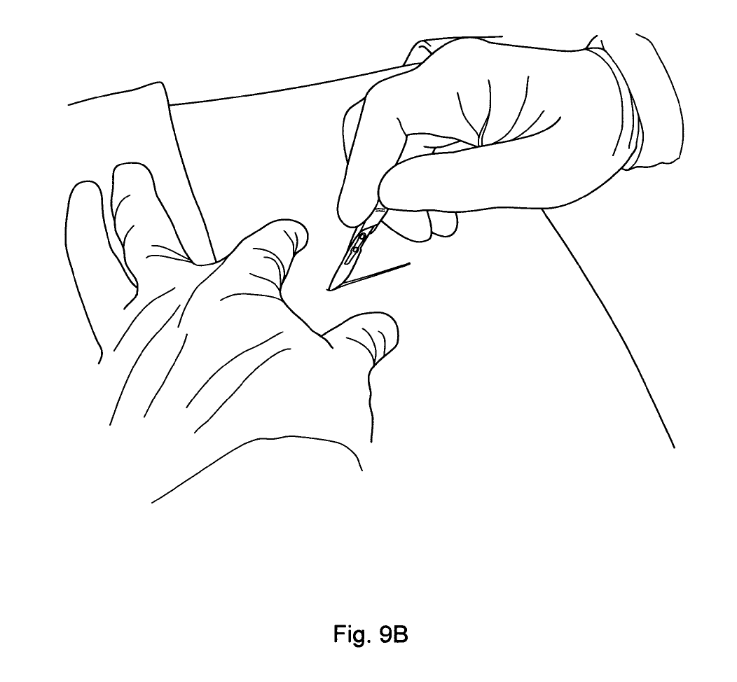

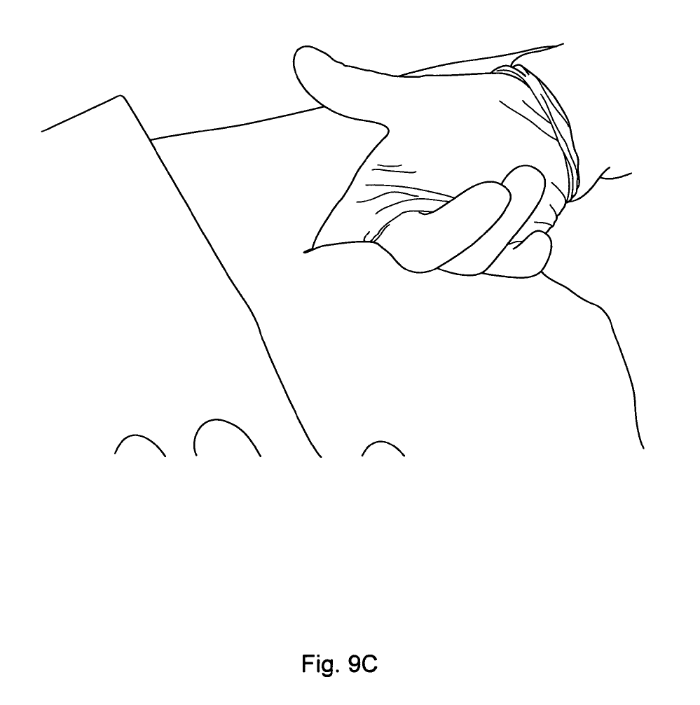

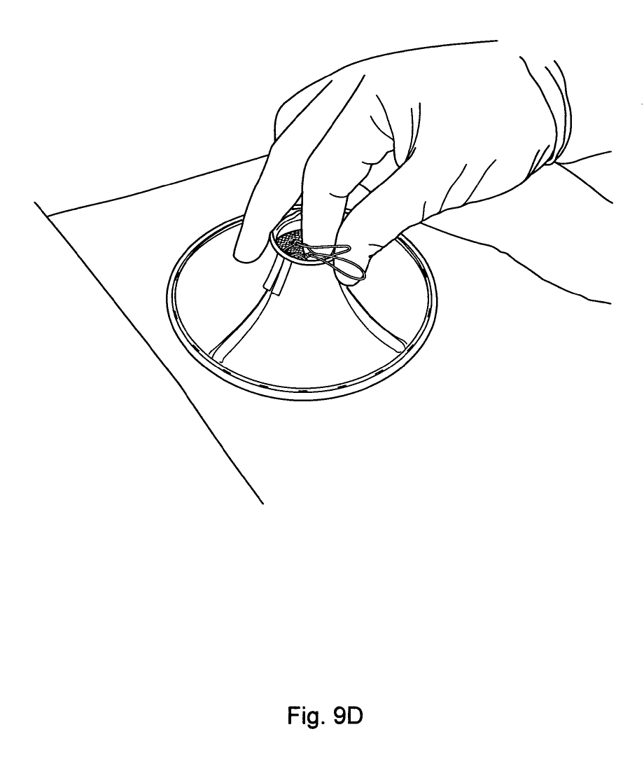

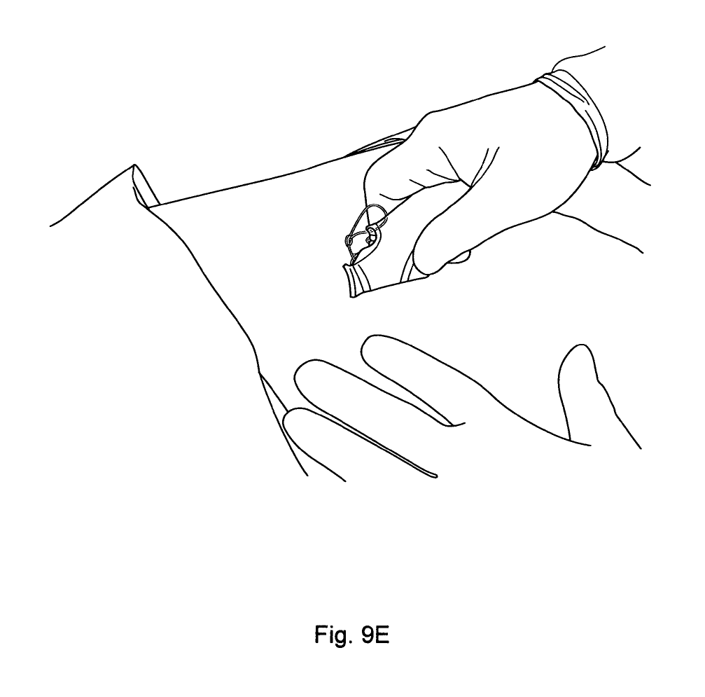

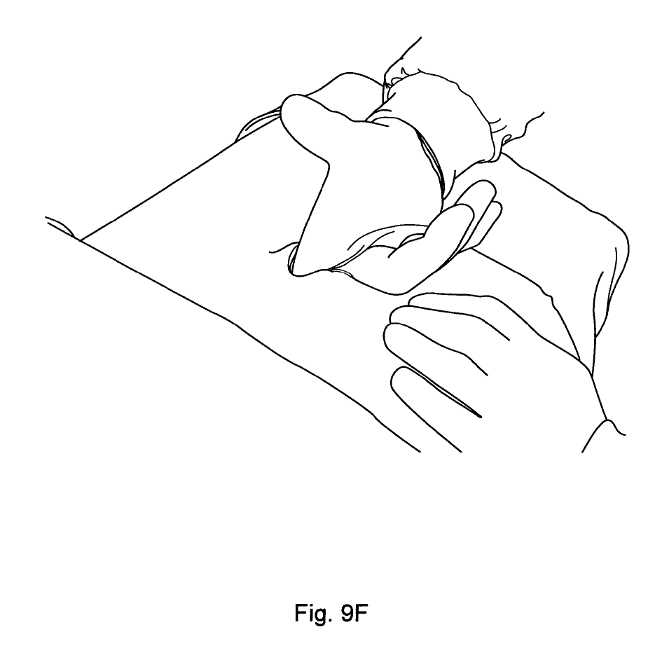

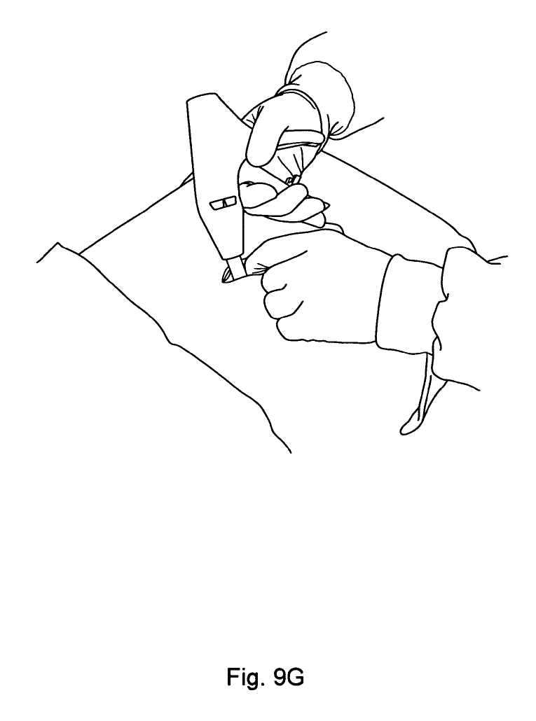

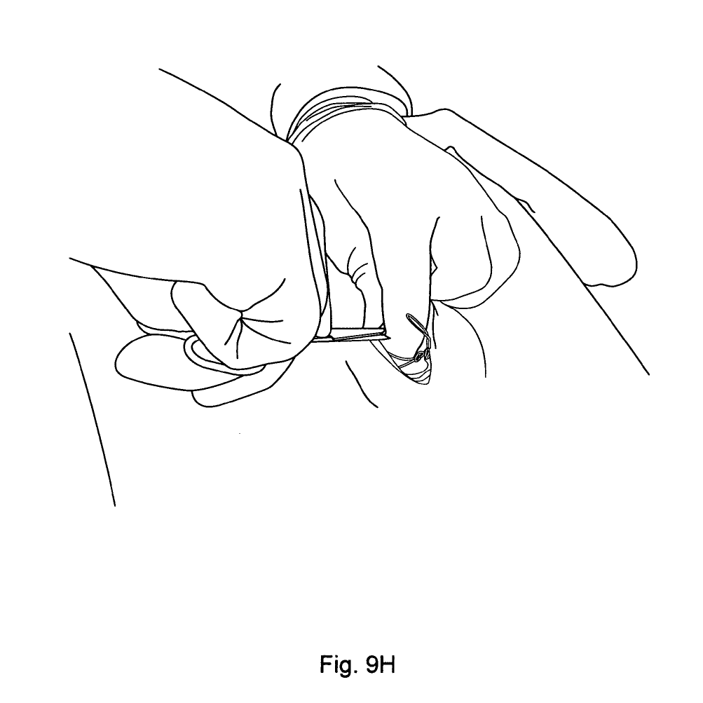

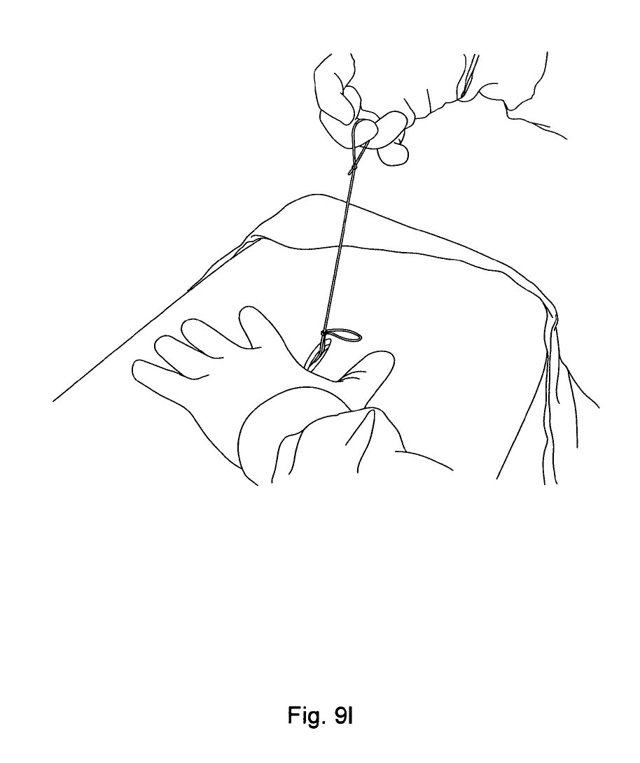

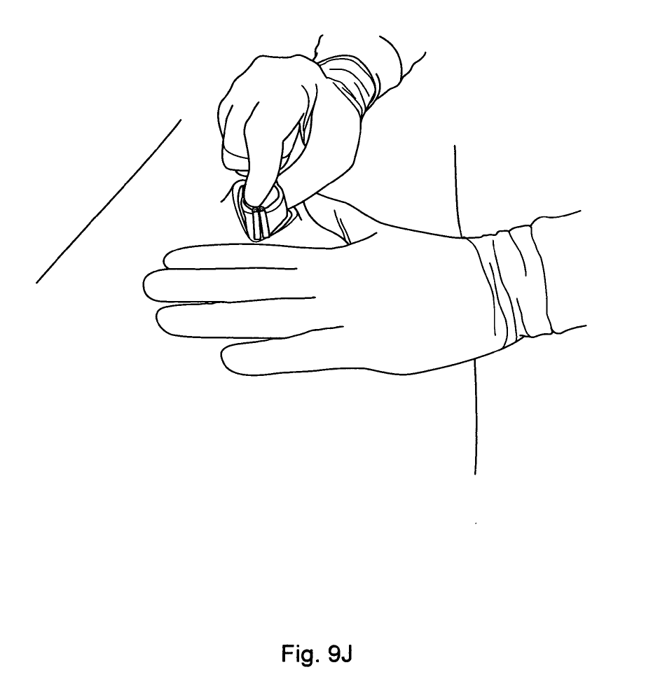

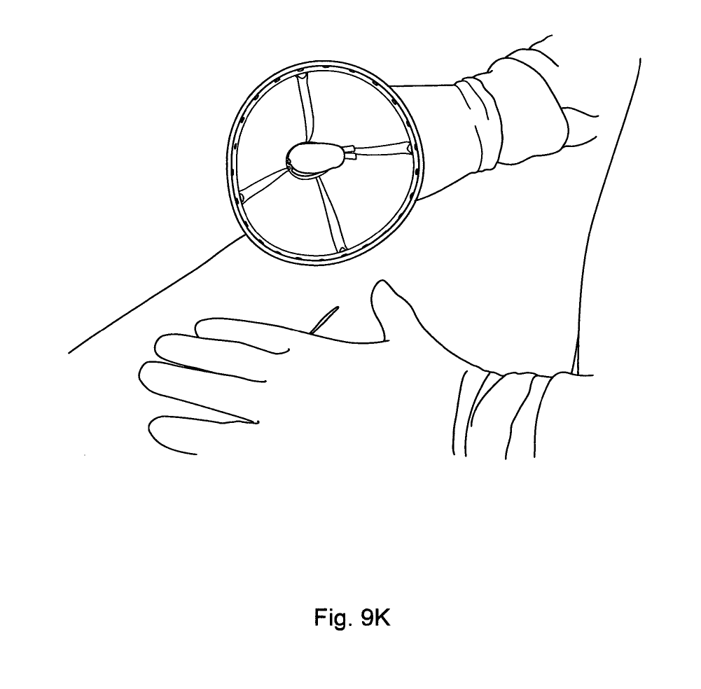

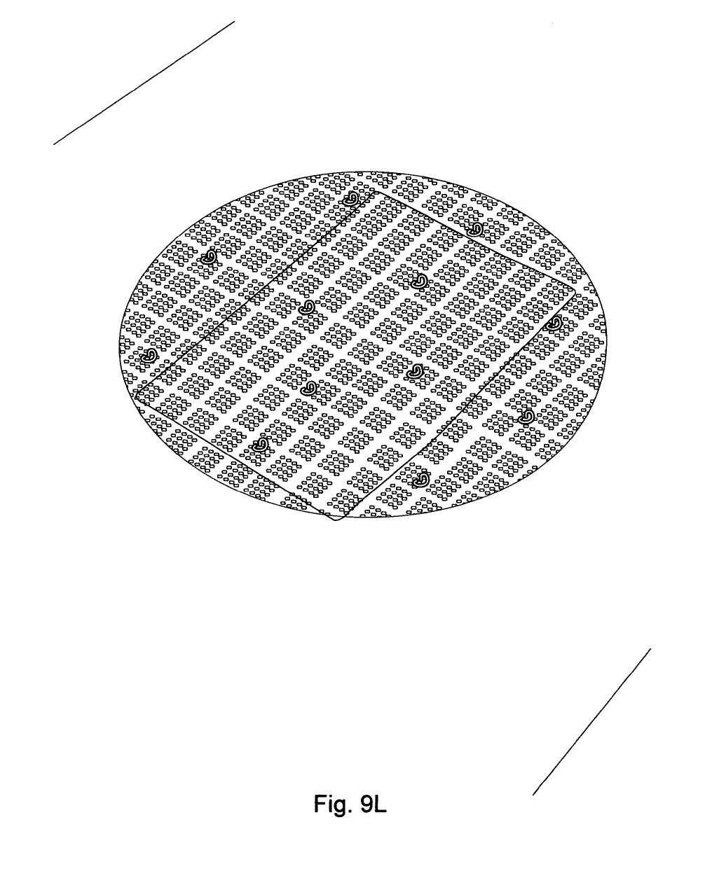

The support element 31 may be located fully within an internal body cavity, as illustrated in FIG. 9F, with the second end 33 of the support element 31 inserted into the internal body cavity. In this position the second end 33 of the support element 31 is engagable with a second portion of body tissue to maintain the second portion of body tissue spaced-apart from the soft tissue implant 16.

The support element 31 comprises an access opening 34 extending at least partially therethrough through which one or more parts may be extended to access the soft tissue implant 16. The part extended through the access opening 34 may be an instrument or a hand/arm of a clinician. The access opening 34 is located at the second end 33 of the support element 31 at the radial centre of the support element 31.

In use, the soft tissue implant 16 is coupled to the support element 31 using the coupling element 22 by extending the coupling element 22 through the openings 35 in the support element 31 and the openings in the soft tissue implant 16.

An incision is made in the abdominal wall (FIGS. 9A to 9C). The support element 31 and the soft tissue implant 16 are collapsed into the delivery configuration and inserted through the opening in the abdominal wall (FIG. 9E) to deliver the soft tissue implant 16 to the desired treatment location. The support element 31 is then released which causes expansion of the soft tissue implant 16 to the deployment configuration (FIG. 9F).

The support element 31 is located fully within the internal body cavity, as illustrated in FIG. 9F, with the second end 33 of the support element 31 inserted into the internal body cavity. In this position the second end 33 of the support element 31 engages with the second portion of body tissue to maintain the second portion of body tissue spaced-apart from the soft tissue implant 16 during deployment of the soft tissue implant 16 at the desired treatment location.

An instrument or hand/arm of a clinician is extended through the access opening 34 to access the soft tissue implant 16 (FIG. 9G). The instrument or clinician's hand is used to attach the soft tissue implant 16 to the first portion of body tissue at the desired treatment location using the attachment element by extending the attachment element through the openings 18. In this manner the first portion of body tissue is treated for example during hernia repair. The support element 31 supports the soft tissue implant 16 during deployment at the desired treatment location.

The support element 31 is then released from the soft tissue implant 16 by removing the coupling element 22 from the openings 35 in the support element 31 and from the openings in the soft tissue implant 16 (FIGS. 9H and 9I). The support element 31 is then removed from the opening in the abdominal wall (FIGS. 9J and 9K).

The support element 31 is configured to be sufficiently flexible to enable the support element 31 to be inserted into the opening in the abdominal wall and removed from the opening in the abdominal wall, and is also configured to have sufficient shape memory to expand to the deployment configuration when released which causes expansion of the soft tissue implant 16.

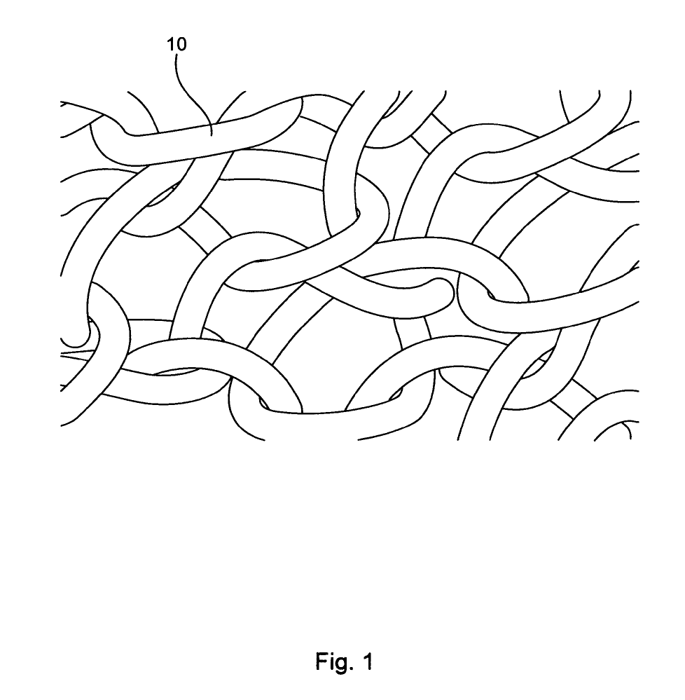

A known non-absorbable mesh implant 10 is illustrated in FIG. 1 using a micrograph.

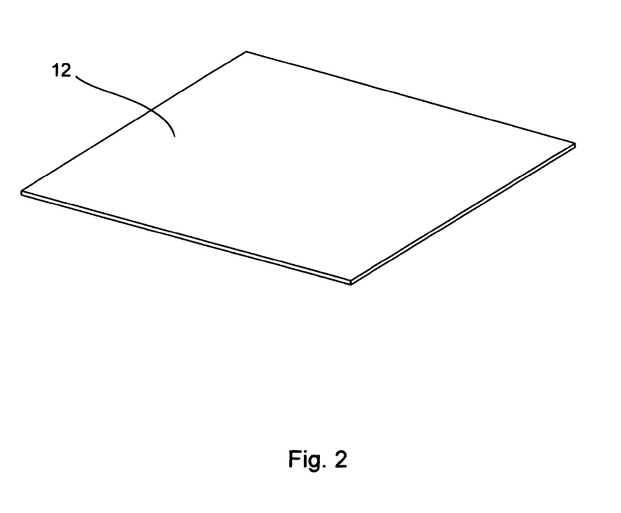

FIG. 2 is a perspective view of the nonwoven biocompatible film 12 used to construct the soft tissue implant. The implant has a known width and length. The biocompatible film 12 is made of a biocompatible material. Biocompatible materials may include non-absorbable polymers (for example polypropylene, polyethyleneterephthalate, polytetrafluoroethylene, polyaryletherketone, nylon, fluorinated ethylene propylene, polybutester, or silicone), absorbable polymers (for example polyglycolic acid, polylactic acid, polycaprolactone, or polyhydroxyalkanoate), or tissue based materials (for example collagen, allograft, or xenograft).

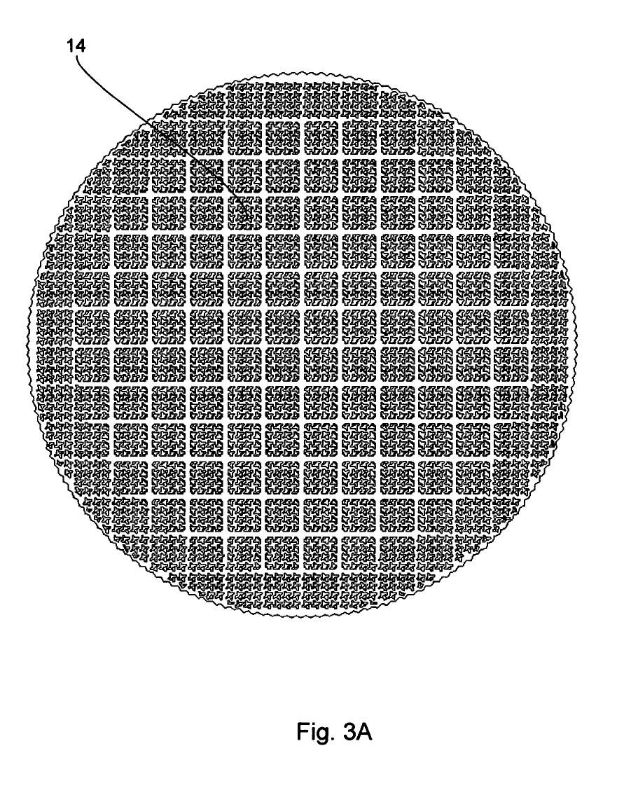

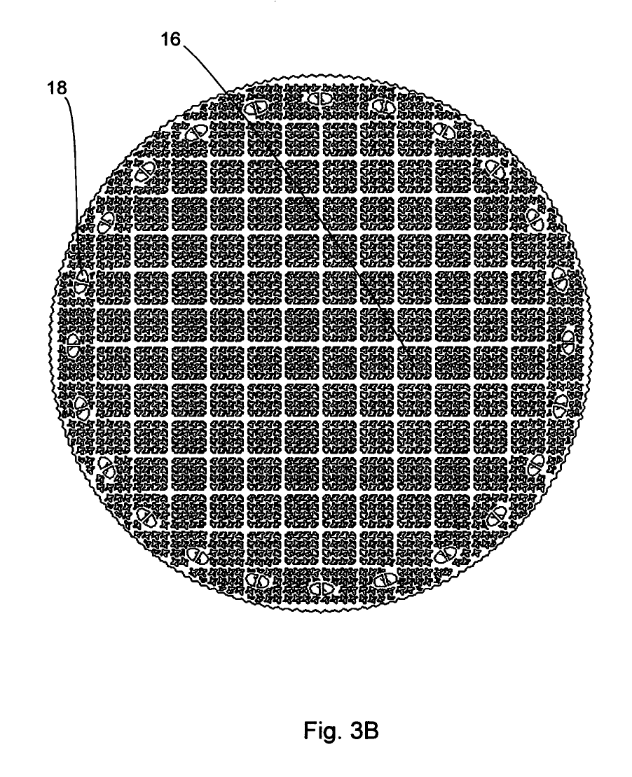

FIG. 3A is a view of a sinusoidal like cell pattern machined in the film 14 with major and minor struts in accordance with the invention. A sinusoidal cell pattern has been machined into the film to impart porosity for tissue ingrowth on high strength thin film substrates. The sinusoidal cell pattern has major and minor struts. Manufacturing methods to impart the cell pattern may include laser machining, die punching, water jet cutting, or chemical etching. The lasers preferred for creating smooth edges on plastic films are CO₂, diode ultraviolet, or excimer lasers. Additional lasers not referenced here may also be acceptable.

FIG. 3B is a view of a sinusoidal like cell pattern machined in the film with delivery system attachment points 18 in accordance with the invention. Attachment points 18 have been machined into the film to facilitate attachment to the delivery means.

FIG. 4A is a perspective view of a minimally invasive delivery system in accordance with the invention. An elastic material with shape memory properties is used to produce the minimally invasive delivery system which comprises a dome 20. The dome 20 is preferably made from a thermoplastic polyurethane that may be moulded. The dome 20 may be of a biological material. The dome 20 may be of a bioabsorbable material. The dome is dimensioned and moulded to approximate the dimensions of the soft tissue implant being used. FIG. 4B is a top view of a minimally invasive delivery system in accordance with the invention. FIG. 4C is a bottom view of a minimally invasive delivery system in accordance with the invention. FIG. 4D is a side view of a minimally invasive delivery system in accordance with the invention.

FIG. 5A is a perspective view of a minimally invasive delivery system which comprises a dome 20 and a soft tissue implant which comprises sinusoidal like cell pattern machined in the film with delivery system attachment points 18 in accordance with the invention. FIG. 5B is a perspective view of a minimally invasive delivery system attached to a soft tissue implant with drawstring attachment means 22 in accordance with the invention. One material suitable for the drawstring 22 is polytetrafluoroethylene due to its lubricious properties.

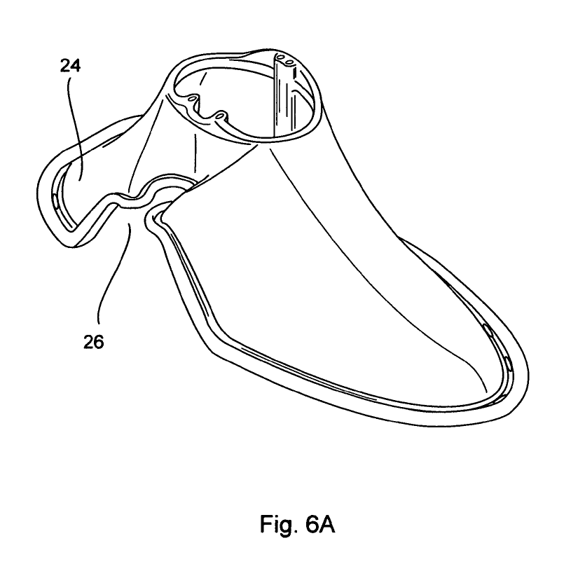

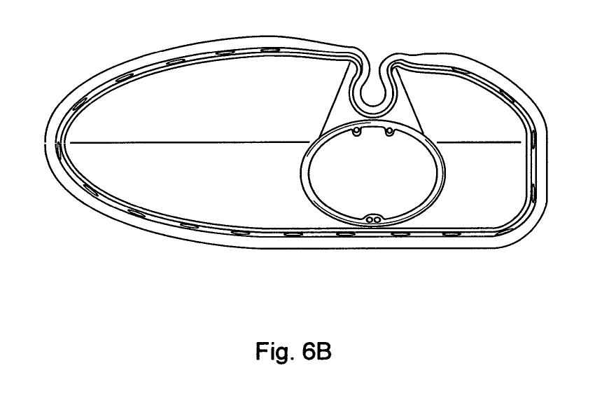

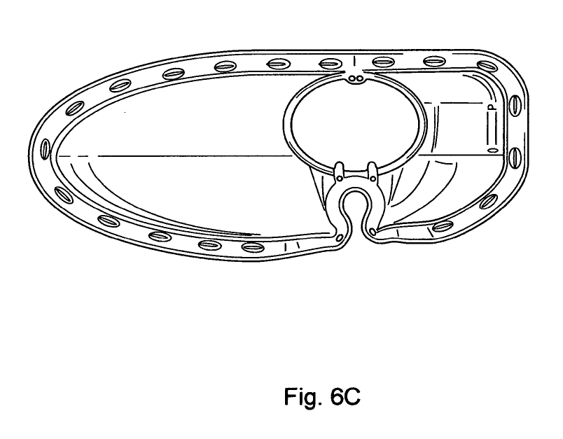

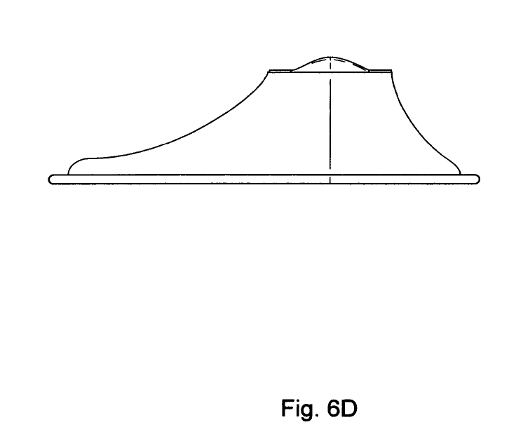

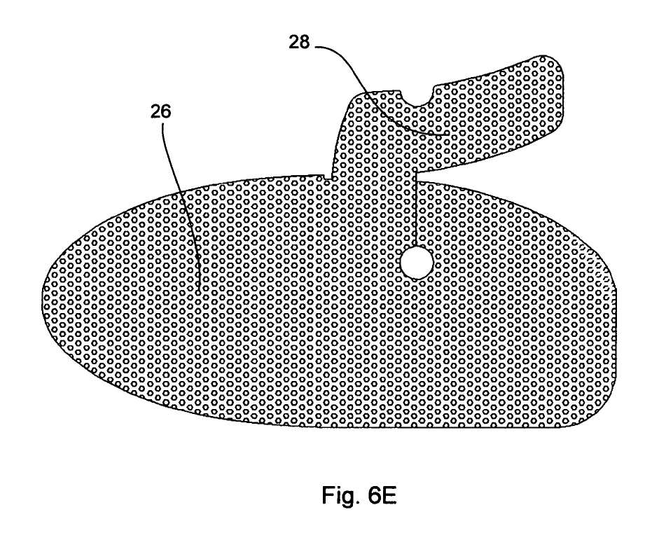

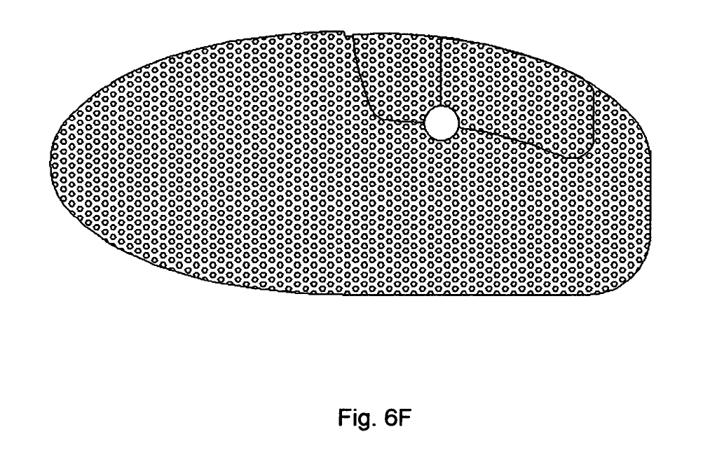

FIG. 6A is a perspective view of a minimally invasive delivery system which comprises a dome 24 designed for inguinal hernia repair. FIG. 6B is a top view of a minimally invasive delivery system in accordance with the invention. FIG. 6C is a bottom view of a minimally invasive delivery system in accordance with the invention. FIG. 6D is a side view of a minimally invasive delivery system in accordance with the invention. FIG. 6E is a plan view of an inguinal soft tissue implant 26 in accordance with the invention with a spermatic cord flap 28. FIG. 6F is a plan view of an inguinal soft tissue implant in accordance with the invention with a spermatic cord flap positioned over the main body of the implant.

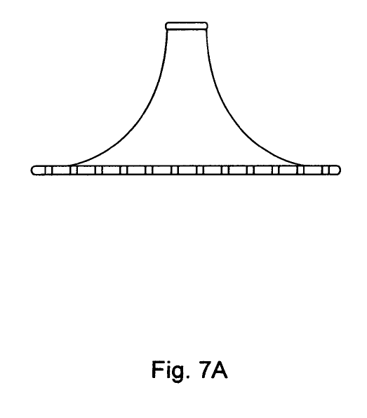

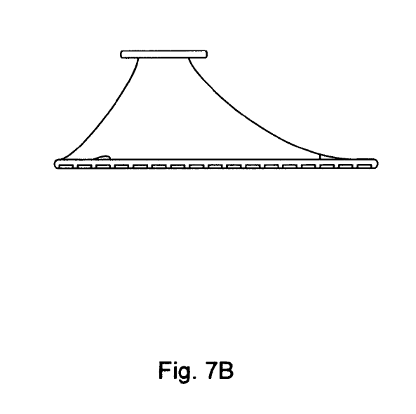

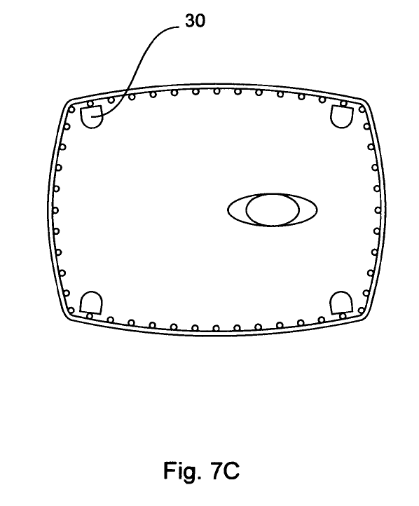

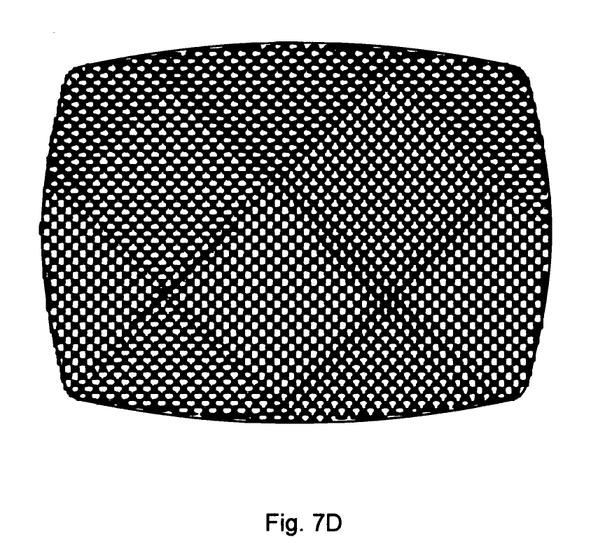

FIG. 7A is a side view of a minimally invasive delivery system in accordance with the invention. FIG. 7B is a side view of a minimally invasive delivery system in accordance with the invention. FIG. 7C is a bottom view of an inguinal soft tissue implant in accordance with the invention. A fixation attachment point 30 is shown. Staples, sutures, or tissue adhesives may be used to secure the implant at the attachment point where the implant is exposed. Alternatively the attachment means may be provided mounted to the implant prior to insertion of the implant, for example barbs, or an adhesive. FIG. 7D is a plan view of an inguinal soft tissue implant in accordance with the invention.

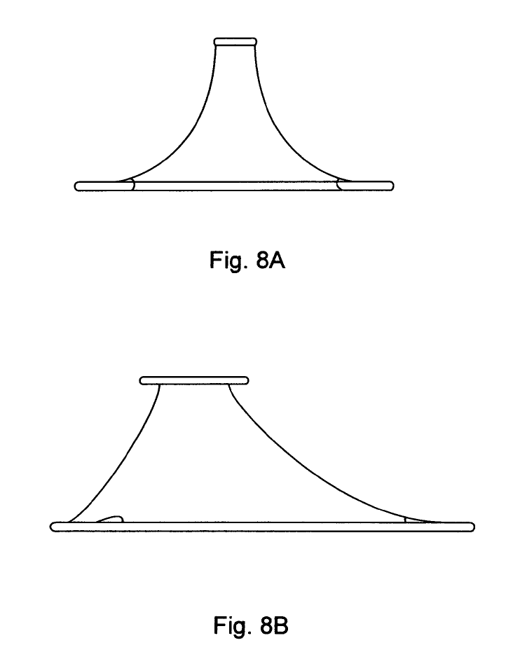

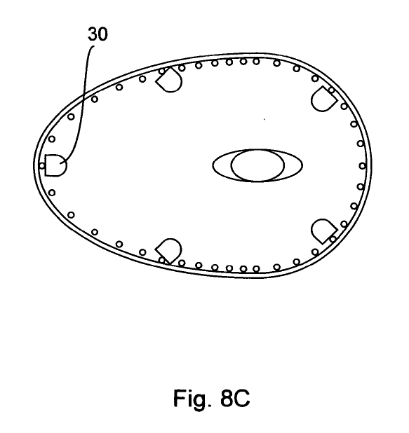

FIG. 8A is a side view of a minimally invasive delivery system in accordance with the invention. FIG. 8B is a side view of a minimally invasive delivery system in accordance with the invention. FIG. 8C is a bottom view of an inguinal soft tissue implant in accordance with the invention. A fixation attachment point 30 is shown. Staples, sutures, or tissue adhesives may be used to secure the implant at the attachment point where the implant is exposed.

FIG. 9A is a photograph of a patient trainer used for surgical training with a 4 cm incision line. FIG. 9B is a photograph of a patient trainer used for surgical training with a scalpel making a 4 cm incision. FIG. 9C is a photograph of a patient trainer used for surgical training with a simulated hernia sac dissection taking place. FIG. 9D is a photograph of a patient trainer used for surgical training with a minimally invasive delivery system in accordance with the invention. FIG. 9E is a photograph of a patient trainer used for surgical training with a minimally invasive delivery system in accordance with the invention being placed over the hernia defect. FIG. 9F is a photograph of a patient trainer used for surgical training with a minimally invasive delivery system in accordance with the invention being expanded to create working space over the hernia defect. FIG. 9G is a photograph of a patient trainer used for surgical training with a minimally invasive delivery system in accordance with the invention. The surgical mesh attached to the minimally invasive delivery system is being stapled to the tissue using a disposable stapler. FIG. 9H is a photograph of a patient trainer used for surgical training with the drawstring being cut on the minimally invasive delivery system in accordance with the invention. FIG. 91 is a photograph of a patient trainer used for surgical training with the drawstring being removed from the minimally invasive delivery system in accordance with the invention. FIG. 9J is a photograph of a patient trainer used for surgical training with the minimally invasive delivery system being removed from the patient in accordance with the invention. FIG. 9K is a photograph of a patient trainer used for surgical training with the minimally invasive delivery system removed from the patient in accordance with the invention. FIG. 9L is a photograph of a patient trainer used for surgical training with the stapled mesh attached to the patient's tissue.

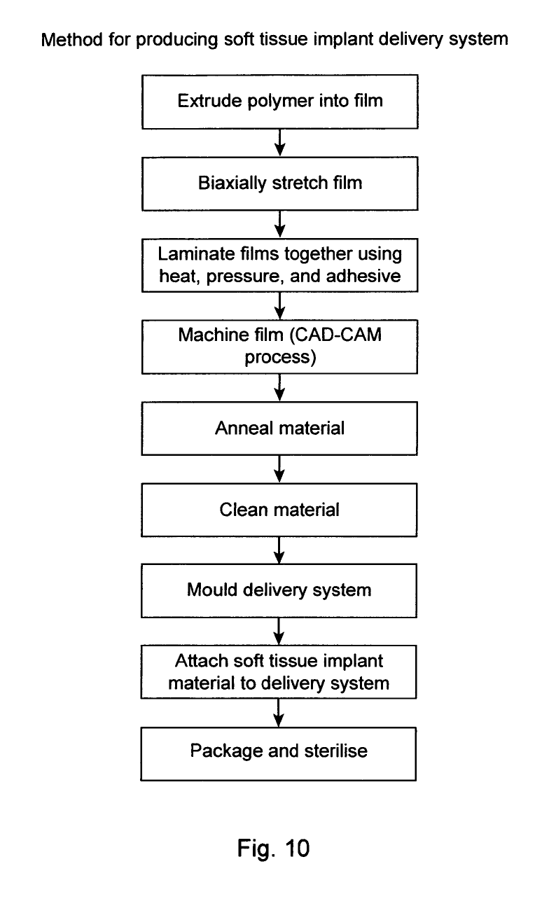

FIG. 10 is a diagram showing the manufacturing steps.

Medical implant applications for the soft tissue implant technology described above may include but are not limited to plastic reconstruction, urinary stress incontinence, hernia repair, chest wall reconstruction, and muscular skeletal defects. The delivery system and soft tissue implant may be produced in a variety of shapes and sizes for the particular indication. A non-absorbable soft tissue implant may be selected for indications such as hernia repair that require long-term durability and strength. An absorbable soft tissue implant may be selected for indications such as tissue augmentation during plastic reconstruction when it is desired to avoid the potential complications associated with a permanent implant. Tissue based materials may be best suited for indications such as pelvic slings that require materials less prone to erosion into adjacent tissue structures.

It will be appreciated that the minimally invasive delivery system and soft tissue implant of this invention may be used to treat bodily defects, may be manufactured with an elastic dome, may be designed in configurations to treat different types of bodily defects, may be designed with flexible soft tissue implants, may be designed with detachable means, may be designed with a working space for the application of sutures, tacks, or tissue adhesives, may be designed for placement and expansion through a small incision, and may be manufactured in a cost effective manner.

Although the description above contains many specifics, these should not be construed as limiting the scope of the invention but as merely providing illustrations of some of the presently preferred embodiments of this invention. For example, the delivery system and implant may have other designs, different materials may be utilised, and alternate equipment may be used to produce the structures.

The invention is not limited to the embodiments hereinbefore described, with reference to the accompanying drawings, which may be varied in construction and detail.

1. A device suitable for use during deployment of a medical device at a desired treatment location, the device comprising a support element to support the medical device during deployment.

2. A device as claimed in claim 1 wherein the support element comprises a first end mounted to a medical device.

3. A device as claimed in claim 2 wherein the support element comprises a second end longitudinally spaced-apart from the first end.

4. A device as claimed in claim 3 wherein the second end of the support element is engagable with a body tissue to maintain the body tissue spaced-apart from a medical device.

5. A device as claimed in claim 1 wherein the support element is configured for location fully within an internal body cavity.

6. A device as claimed in claim 3 wherein the second end of the support element is insertable into an internal body cavity.

7. A device as claimed in claim 3 wherein the support element tapers inwardly from the first end towards the second end.

8. A device as claimed in claim 1 wherein the support element is curved in longitudinal cross-section.

9. A device as claimed in claim 8 wherein the concave portion of the curve faces radially outwardly.

10. A device as claimed in claim 1 wherein the support element is substantially dome-shaped.

11. A device as claimed in claim 1 wherein the support element comprises an access opening extending at least partially therethrough through which one or more parts may be extended to access a medical device.

12. A device as claimed in claim 11 wherein the access opening is located substantially at the second end of the support element.

13. A device as claimed in claim 11 wherein the access opening is located substantially at the radial centre of the support element.

14. A device as claimed in claim 1 wherein the device comprises a coupling element to couple a medical device to the support element.

15. A device as claimed in claim 14 wherein the coupling element is configured to releasably couple a medical device to the support element.

16. A device as claimed in claim 14 wherein the coupling element is extendable through an opening in the support element.

17. A device as claimed in claim 14 wherein the coupling element is extendable through an opening in a medical device.

18. A device as claimed in claim 14 wherein the coupling element is substantially elongate.

19. A device as claimed in claim 18 wherein the coupling element comprises a drawstring.

20. A device as claimed in claim 14 wherein the coupling element comprises a low-friction material.

21. A device as claimed in claim 1 wherein the support element is movable between a delivery configuration for delivery of a medical device to a desired treatment location, and a deployment configuration for deployment of the medical device at the desired treatment location.

22. A device as claimed in claim 21 wherein the support element is collapsed in the delivery configuration.

23. A device as claimed in claim 21 wherein the support element is expanded in the deployment configuration.

24. A device as claimed in claim 21 wherein the support element is biased towards the deployment configuration.

25. A device as claimed in claim 1 wherein the support element comprises a resilient material.

26. A device as claimed in claim 1 wherein the support element comprises a shape-memory material.

27. A device as claimed in claim 1 wherein the support element comprises a thermoplastic material.

28. A device suitable for use during deployment of a medical device at a desired treatment location substantially as hereinbefore described with reference to the accompanying drawings.

29. An assembly comprising:
a medical device; and
a device as claimed in claim 1 for use during deployment of the medical device at a desired treatment location.

30. An assembly as claimed in claim 29 wherein the medical device comprises an implant.

31. An assembly as claimed in claim 30 wherein the medical device comprises a soft tissue implant.

32. An assembly as claimed in claim 29 wherein the medical device is configured to be attached to a body tissue at a desired treatment location.

33. An assembly as claimed in claim 32 wherein the medical device comprises one or more openings through which an attachment element may be extended.

34. An assembly as claimed in claim 29 wherein the medical device comprises one or more openings through which the coupling element is extendable.

35. An assembly substantially as hereinbefore described with reference to the accompanying drawings.

36. A method of deploying a medical device at a desired treatment location, the method comprising the steps of:
using a support element to support the medical device during deployment at the desired treatment location,
attaching the medical device to a first body tissue at the desired treatment location, and removing the support element from the desired treatment location.

37. A method as claimed in claim 36 wherein during deployment of the medical device at the desired treatment location, the support element engages with a second body tissue to maintain the second body tissue spaced-apart from the medical device.

38. A method as claimed in claim 36 wherein one or more parts are used to attach the medical device to the first body tissue.

39. A method as claimed in claim 38 wherein the method comprises the step of extending the part at least partially through the support element to access the medical device.

40. A method as claimed in claim 36 wherein the method comprises the step of coupling the medical device to the support element before delivery to the desired treatment location.

41. A method as claimed in claim 40 wherein the method comprises the step of de-coupling the medical device from the support element after attaching the medical device to the first body tissue.

42. A method as claimed in claim 36 wherein the method comprises the step of collapsing the medical device before delivery to the desired treatment location.

43. A method as claimed in claim 42 wherein the method comprises the step of expanding the medical device before attaching to the first body tissue.

44. A method of deploying a medical device at a desired treatment location substantially as hereinbefore described with reference to the accompanying drawings.

45. A soft tissue implant for repairing a bodily defect comprising:
a first shape memory delivery system component sized and shaped to extend beyond the bodily defect in a patient, the delivery system having a domed configuration and having a selected elasticity,
a biocompatible soft tissue implant; and
an attachment means to the soft tissue implant, the attachment means being reversibly attached to the soft tissue implant.
